(19) United States
(12) Patent Application Publication
(10) Pub. No.: US 2025/0127957 A1
(43) Pub. Date: Apr. 24, 2025

(54) ADDITIVE MANUFACTURING METHOD FOR PRODUCING BIO-MIMETIC NANOCOMPOSITE SCAFFOLD

(71) Applicant: Brinter Inc., Santa Monica, CA (US)

(72) Inventors: Tomi Alapaattikoski, Santa Monica, CA (US); Eva Mueller, Santa Monica, CA (US); Matthew Brovold, Santa Monica, CA (US); Tomi Kalpio, Santa Monica, CA (US); Dhayakumar Rajan Prakash, Santa Monica, CA (US)

(73) Assignee: Brinter Inc., Santa Monica, CA (US)

(21) Appl. No.: 18/915,523

(22) Filed: Oct. 15, 2024

(30) Foreign Application Priority Data
Oct. 20, 2023 (FI) 20236166

(51) Int. Cl. A61L 27/26; A61L 27/38; A61L 27/54; B33Y 10/00; B33Y 50/00; B82Y 30/00; C08L 5/08; C08L 89/06

(52) U.S. Cl. CPC A61L 27/26; A61L 27/3817; A61L 27/54; B33Y 10/00; B33Y 50/00; C08L 5/08; C08L 89/06; A61L 2300/252; A61L 2400/12; A61L 2430/06; B82Y 30/00

(57) ABSTRACT

Disclosed is an additive manufacturing method for producing a bio-mimetic nanocomposite scaffold using a multi-dimensional printer. The method includes generating a bio-mimetic tool path for a given connective tissue; receiving a bio-mimetic nanocomposite printable material into one or more printing heads of the multi-dimensional printer; and dispensing the bio-mimetic nanocomposite printable material in one or more layers on a printing surface, based on the generated bio-mimetic tool path, for producing the bio-mimetic nanocomposite scaffold.

### TECHNICAL FIELD

The present disclosure relates to additive manufacturing methods for producing bio-mimetic nanocomposite scaffolds using multi-dimensional printers.

### BACKGROUND

In recent years, additive manufacturing has emerged as a technique with immense potential in the field of bioprinting. In this regard, the principles of traditional additive manufacturing are combined with advanced bio-fabrication techniques to enable a precise deposition of biomaterials, cells and other bioactive components to create complex tissue structures.

Conventionally, 3D bioprinting is used for fabricating three-dimensional tissue constructs for both improved in vitro testing and tissue/organ replacements. However, existing bioprinting processes exhibit shortcomings, particularly in yielding constructs with inadequate mechanical features, thus limiting their efficacy for fabricating functional cell-laden tissues or augmenting the body's native regenerative capabilities. Notably, bioprinting using the additive manufacturing bridges a divide between two-dimensional cell culture and in vivo experiments by facilitating a simultaneous deposition of biomaterials, cells and other bioactive components in a single printing step into a pre-defined three-dimensional construct. However, despite the vast potential of using 3D bioprinting as an effective additive manufacturing process for fabricating functional soft tissue constructs, clinical translation is limited due to the inability to mimic the mechanics and native tissue microenvironment.

Therefore, in light of the foregoing discussion, there exists a need to overcome the aforementioned drawbacks.

### SUMMARY

The aim of the present disclosure is to provide an additive manufacturing method for producing functional and bio-mimetic tissue engineered scaffolds, also known as bio-implants, for connective tissue present in a subject. The aim of the present disclosure is achieved by an additive manufacturing method for producing a bio-mimetic nanocomposite scaffold using a multi-dimensional printer as defined in the appended independent claims to which reference is made to. Advantageous features are set out in the appended dependent claims.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIG. 1 is an illustration of a flowchart depicting steps of an additive manufacturing method for producing a bio-mimetic nanocomposite scaffold using a multi-dimensional printer, in accordance with an embodiment of the present disclosure;

FIG. 2A is an illustration of a scan of a given connective tissue of a knee of a subject, in accordance with an embodiment of the present disclosure;

FIG. 2B is an illustration of a scan of a microscopic architecture of the given connective tissue of the subject, in accordance with an embodiment of the present disclosure;

FIG. 2C is an illustration of an orientation of nanofibers in the given connective tissue of the subject, in accordance with an embodiment of the present disclosure;

FIGS. 3A and 3B is an illustration of a printing head of a multi-dimensional printer performing an additive manufacturing, in accordance with an embodiment of the present disclosure; and

FIG. 4 is an illustration of a process of generating a bio-mimetic tool path for a given connective tissue, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In a first aspect, the present disclosure provides an additive manufacturing method for producing a bio-mimetic nanocomposite scaffold using a multi-dimensional printer, the method comprising:
generating a bio-mimetic tool path for a given connective tissue;
receiving a bio-mimetic nanocomposite printable material into one or more printing heads of the multi-dimensional printer; and
dispensing the bio-mimetic nanocomposite printable material in one or more layers on a printing surface, based on the generated bio-mimetic tool path, for producing the bio-mimetic nanocomposite scaffold.

The first aspect of the disclosed additive manufacturing method enables the precise fabrication of the bio-mimetic nanocomposite scaffolds using the multi-dimensional printer, allowing for tailored solutions in tissue engineering and regenerative medicine. The method enables generating the bio-mimetic tool path that ensures that both the macroscopic dimensions of the construct (typical for 3D printing/additive manufacturing solutions) and the microscopic ECM architecture (through the use of a pre-defined g-codes/infill pattern) are captured for the fabricated tissue construct, enhancing the bio-mimicry to native tissue and enables improvement in cellular responses and mechanical strength. Moreover, the method enables selecting the bio-mimetic nanocomposite printable materials that are both biocompatible and biodegradable with sufficient bulk physical properties for the target tissue engineering application. Furthermore, the method enables dispensing the bio-mimetic nanocomposite printable material based on the bio-mimetic tool path. It will be appreciated that the aforementioned steps work synergistically to promote tissue regeneration and repair, making it a promising advancement in the field of biomedical engineering with the potential to revolutionize personalized medicine and tissue engineering applications.

Throughout the present disclosure, the term “additive manufacturing” as used herein refers to a process of dispensing a printable material in a layer-by-layer fashion to fabricate an object with multiple dimensions. Generally, the additive manufacturing includes a three-dimensional (3D) printing which is the process of joining materials to make parts from 3D model data, typically in a layer upon layer manner. Moreover, the additive manufacturing finds application in 3D bioprinting which is an automated deposition process controlled by a CAD model. Different to the conventional plastics or metal additive manufacturing, the 3D bioprinting allows for the printing of both biomaterials and biologically functional constituents, such as cells, in a single step and in a pre-defined spatial arrangement, essentially printing living structures layer by layer.

Furthermore, the additive manufacturing includes a four-dimensional (4D) printing that uses the same techniques as described in the 3D printing through computer-programmed deposition of materials to create a 4D object or construct layer-by-layer. Additionally, the 4D bioprinting enables the precise deposition of biomaterials and biologically active components that can undergo structural deformations over time. These time-dependent changes allow for encapsulated components to fold or unfold, encapsulating and releasing drugs or cells in a programmable process. Furthermore, the additive manufacturing includes a five-dimensional (5D) printing, a six-dimensional (6D) printing, and so forth based on time, axes, and stimulus required when in operation. Moreover, the additive manufacturing includes volumetric printing. In this regard, the volumetric printing allows a simultaneous creation of the 3D object by irradiating a volume of photosensitive resin from multiple angles. Vat polymerisation uses a vat of liquid photopolymer resin, out of which the model is constructed layer by layer. An ultraviolet (UV) light is used to cure or harden the resin where required, whilst a platform moves the object being made downwards after each new layer is cured. Herein, bioprinting refers to a form of additive manufacturing to produce a three-dimensional biological construct. The term “bio-mimetic nanocomposite scaffold” as used herein refers to a three-dimensional structure or framework composed of a bulk hydrogel combined with ECM-like components to mimic the native composition of native functional cartilage tissues.

In this regard, the cartilage tissues include articular cartilage, hyaline cartilage, elastic cartilage, fibrocartilage, and so forth. The articular cartilage is a specialized type of cartilage found at the ends of bones in various joints throughout the human body, including the knee, hip, shoulder, elbow, ankle, wrist, and sternoclavicular joints. The articular cartilage is a smooth, white tissue that serves crucial functions, such as reducing friction between bones during movement, absorbing shocks, and distributing loads evenly across the joint. The articular cartilage acts as a protective cushion, allowing for pain-free and smooth articulation between bones while ensuring joint health and function. The hyaline cartilage is a type of connective tissue found in areas requiring smooth joint movement, such as the ends of long bones, rib cartilage, larynx, trachea and the nose. Moreover, the hyaline cartilage provides structural support while allowing flexibility due to its glassy appearance and high collagen content. The elastic cartilage contains more elastic fibers, making it extremely flexible and found in structures such as the ear, auricles, eustachian tube, laryngeal cartilages and the epiglottis, providing both shape and flexibility. The fibrocartilage is a connective tissue, rich in collagen, and found in areas such as intervertebral discs, pubic symphysis, manubriosternal joint, shoulder joint, hip joint, menisci of the knee joint and location where tendons and ligaments attach to bone, offering both support and shock absorption.

It will be appreciated that the bio-mimetic nanocomposite scaffold is designed to mimic the natural tissue environment found in cartilage, providing a suitable microenvironment for cell adhesion, proliferation, and tissue regeneration. In this regard, the additive manufacturing is performed using a multi-dimensional printer. The term “multi-dimensional printer” (namely, a multi-material printer) as used herein refers to an additive manufacturing device for dispensing materials of different dimensions in a single processing step. The multi-dimensional printer comprises various components such as, a printing surface, one or more printing heads and has multiple configurable axes such as linear or rotational. Optionally, the multi-dimensional printer comprises a printing chamber. The components, shape, size, and design of the multi-dimensional printer may vary according to the bioprinting application thereof.

The term “bio-mimetic tool path” as used herein refers to a digital path or trajectory that guides the deposition or placement of materials within the multi-dimensional printer during the fabrication process. Advantageously, the bio-mimetic tool path is designed to replicate or mimic the natural or native architecture of the extracellular matrix within the target connective tissue. The term “given connective tissue” as used herein refers to a biological tissue or organ of interest that is selected or designated for replication, analysis, or manipulation thereof. Typically, the connective tissues are a diverse group of tissues found in the human body that provide structural support, connect different body parts, and perform various functions. The method comprises generating the bio-mimetic tool path for the given connective to mimic or replicate the natural architecture and features of the bio-mimetic nanocomposite scaffold being printed. Advantageously, said bio-mimicking enhances regenerative capabilities and compatibility of the bio-mimetic nanocomposite scaffold with the body. The bio-mimetic tool path guides the deposition of the bio-mimetic nanocomposite printable material, layer by layer, in a precise and biomimetic manner. Moreover, an advanced computer-aided design (CAD) software implemented on the multidimensional printer is configured to analyse the anatomical data and generate the bio-mimetic tool path. The bio-mimetic tool path guides the one or more printing heads of the multidimensional printer. The three main types of connective tissue are loose connective tissue, dense connective tissue, and specialized connective tissue. In this regard, the loose connective tissue is characterized by its loose arrangement of collagen and elastic fibers, and it surrounds organs and provides flexibility. The dense connective tissue, is densely packed with collagen fibers, offering exceptional strength and durability. Moreover, the dense connective tissue is found in tendons and ligaments. The specialized connective tissue includes a variety of subtypes such as the cartilage, the bone, and the blood, each with its unique properties and functions. The cartilage provides firmness and flexibility, bone offers rigidity and support, and blood connects body tissues while serving as a medium for nutrient and waste transport.

Optionally, generating the bio-mimetic tool path comprises performing at least one of: an anatomical sectioning of given connective tissue using a biopsy, a real-time imaging using collagen auto-fluorescence. The term “anatomical sectioning” as used herein refers to a technique used to cut or slice a biological tissue sample into thin, sequential sections, typically for detailed microscopic analysis or to gather information about the geometry, structure, composition, dimensions, and other relevant characteristics thereof. The term “biopsy” as used herein refers to a medical procedure in which a small sample of tissue or cells is removed from a living organism for examination and analysis. In this regard, by performing anatomical sectioning, anatomical data having three-dimensional structural information about the given connective tissue at a microscopic level is obtained. Optionally, the anatomical data could be acquired through various medical imaging techniques such as Computed Tomography (CT) scans, magnetic resonance imaging (MRI), ultrasound, or 3D scans, providing a comprehensive representation of the tissue's internal and external features.

The term “real-time imaging using collagen auto-fluorescence” as used herein refers to a technique that involves capturing images of the given connective tissue by exploiting the natural fluorescence emitted by collagen fibers when exposed to specific wavelengths of light. In such a case, the excitation light has a wavelength range of 270-370 nanometers (nm), which is used to stimulate the collagen fibers. Moreover, when the collagen fibers are excited within said range, the collagen fibers absorb this energy and then emit light in response, with a fluorescence peak typically occurring between 305-450 nm. Optionally, a specialized imaging equipment, such as a confocal microscope or a multi-photon microscope, is used to visualize the given connective tissue. The emitted signals are then detected and used to create detailed images of the tissue's collagen network. Beneficially, the real-time imaging using collagen auto-fluorescence can aid in rapidly capturing data about collagen orientation and distribution. The aforementioned two sets of data are integrated and analyzed using a specialized software. Optionally, the software (such as a g-code) uses the aforementioned data to generate the bio-mimetic tool path. Optionally, the real-time imaging could be performed with, but not limited to extracellular matrix proteins including non-collagen and proteoglycans.

The term “bio-mimetic nanocomposite printable material” as used herein refers to a substance or formulation containing nano and micron-sized fibres that could emulate an intricate structure of the given connective tissue and is capable of being deposited onto the printing surface. The method comprises receiving the bio-mimetic nanocomposite printable material into the one or more printing heads of the multidimensional printer. Optionally, the number of the one or more printing heads being used is selected according to the application thereof.

It will be appreciated that the bio-mimetic nanocomposite printable material could be used as a bio-ink in multidimensional bioprinters for tissue engineering and regenerative medicine. Optionally, the bio-mimetic nanocomposite printable material could be used for drug screening and testing thus leading to improved outcomes and better healthcare solutions for a subject (namely, a patient). Herein, the term “tissue regeneration” refers to a natural process by which damaged or injured tissues in a body of the subject are replaced and repaired through the recruitment or in-growth of cells and/or the expansion and differentiation of resident stem cells which in turn repair and replace the tissue. In an implementation, the tissue regeneration allows the body to heal and restore its functionality after an injury or a disease.

Optionally, receiving the bio-mimetic nanocomposite printable material comprises a mixture a bulk polymer crosslinked matrix with a nanomaterial to form the bio-mimetic nanocomposite printable material. The term “bulk polymer crosslinked matrix” as used herein refers to a concentrated volume >80% v/v of the bio-mimetic nanocomposite printable material used as a main component or base material for the tissue regeneration. Herein, the term “hydrogel” refers to a three-dimensional network of hydrophilic polymers that are capable of absorbing and retaining large amounts of water. Optionally, the hydrogels could be crosslinked either via a physical bond formed by molecular entanglements, and/or secondary forces including ionic, H-bonding or hydrophobic forces (such as via hydrogen bonding, and/or ionic crosslinking) or a chemical bond (such as via photocrosslinking, covalent bond formation, and/or click chemistry). In this regard, the bulk polymer crosslinked matrix is a hydrogel formulation consisting of natural polymers derived from natural sources, such as proteins or carbohydrates.

Optionally, the bulk polymer crosslinked matrix comprises: collagen, gelatin, chitosan, agarose, alginate, fibronectin, cellulose, glycosaminoglycans (GAG), deoxyribonucleic acid (DNA), adhesion glycoproteins, elastin, or a combination thereof. The term “collagen” as used herein refers to a naturally occurring protein found in connective tissues and is biocompatible with the body of the subject. Collagen can be extracted and purified from a variety of sources and offers low immunogenicity, a porous structure, good permeability, biocompatibility and biodegradability. Collagen scaffolds have been widely used in tissue engineering due to these excellent properties. Advantageously, the collagen promotes cell adhesion and regeneration when used as the bulk polymer crosslinked matrix. Typically, the collagen facilitates a controlled release of bioactive substances due to a porous structure thereof. Beneficially, the collagen is beneficial in wound healing and drug delivery systems.

The term “gelatin” as used herein refers to a derivative of collagen, often used in biomedical applications for its gel-forming properties. Advantageously, the gelatin has many advantages, such as swelling capacity, biodegradability, biocompatibility, and commercial availability. The gelatin is widely used in the field of pharmacy, medicine, and the food industry. Beneficially, the gelatin solutions easily form hydrogels during cooling. The term “chitosan” as used herein refers to a biopolymer derived from chitin, used for its biocompatibility and wound healing properties. The term “agarose” as used herein refers to a polysaccharide derived from seaweed. The term “alginate” as used herein refers to a natural polysaccharide obtained from brown algae, frequently used in tissue engineering for its biocompatibility and ability to form gels. The term “fibronectin” as used herein refers to a glycoprotein involved in cell adhesion and tissue development and repair. The term “cellulose” as used herein refers to a plant-derived polysaccharide. The term “glycosaminoglycans” (GAG) as used herein refers to long chains of carbohydrates found in the connective tissues, for maintaining tissue structure. The term “deoxyribonucleic acid” (DNA) as used herein refers to the genetic material of cells, carrying genetic instructions for growth, development, and functioning. The term “adhesion glycoproteins” as used herein refers to proteins that promote cell adhesion to extracellular matrix components, facilitating tissue formation. The term “elastin” as used herein refers to a protein that provides elasticity to tissues like skin and blood vessels. In this regard, the bulk polymer crosslinked matrix can consist of one or more of the aforementioned substances, providing flexibility and adaptability to suit different tissue engineering applications.

Optionally, the collagen is selected from: collagen I, collagen II, collagen III, collagen IV, collagen V, collagen VI, or collagen XVIII. In this regard, the collagen I and the collagen II provide mechanical support and contribute to the organization of the tissue. In an example, the collagen I and the collagen II are structural proteins found in connective tissues like cartilage of the body. The collagen III is a fibrillar collagen involved in tissue repair and found in the connective tissues. Optionally, the collagen III is selected for its role in healing and tissue regeneration applications. The collagen IV is a major component of basement membranes in various tissues. Optionally, the collagen IV is selected for its role in providing structural support to the connective tissues. The collagen V is a fibrillar collagen often associated with the collagen I. Optionally, the collagen V is selected for its potential to enhance the structural integrity of bio-mimetic scaffolds. The collagen VI is a critical component of extracellular matrices in tissues like muscles. Optionally, the collagen VI is selected to mimic the complex microenvironment of the connective tissues. The collagen XVIII is known for its role in angiogenesis and tissue development. Optionally, the collagen XVIII is selected to promote vascularization and tissue integration within the bio-mimetic nanocomposite scaffold.

Optionally, the glycosaminoglycans are linear polysaccharides consisting of repeating disaccharide units, including but not limited to hyaluronic acid, chondroitin-6-sulfate, chondroitin-4-sulfate, or keratin-sulphate. The term “hyaluronic acid” as used herein refers to a naturally occurring polysaccharide having a high water-binding capacity. It will be appreciated that the hyaluronic acid when used as the nanomaterial, form hydrogels or nanofibers that offer biocompatibility and aid in the tissue regeneration and wound healing. The term “chondroitin-6-sulfate” is a glycosaminoglycan often found in cartilage and connective tissues. The chondroitin-6-sulfate can be incorporated into the scaffold's formulation to enhance its structural integrity and support tissue regeneration, particularly in cartilage repair applications. The term “chondroitin-4-sulfate” as used herein refers to another glycosaminoglycan present in various tissues, including cartilage. The chondroitin-4-sulfate is used in the bio-mimetic nanocomposite scaffold to contribute to its biomechanical properties and promote the regeneration of tissues with a chondroitin-rich extracellular matrix. The term “keratin-sulfate” as used herein refers to a component of keratin, a protein found in hair, skin, and nails. Optionally, the keratin-sulfate is included in the bulk polymer crosslinked matrix for its potential to support tissue growth and mimic the microenvironment of certain tissues, such as skin or hair follicles.

The term “nanomaterial” as used herein refers to a material composed of nano-sized particles of different shapes or fibers that are incorporated into the bulk polymer crosslinked matrix to create the bio-mimetic nanocomposite printable material. It will be appreciated that the nanomaterials enhance the mechanical strength, structural integrity, and bioactivity of the bio-mimetic nanocomposite material. Notably, the nanomaterials offer a large surface area-to-volume ratio, providing ample sites for the attachment and interaction of the biomolecules, growth factors, and/or peptide sequences that promote the tissue regeneration and cellular responses. Moreover, the nanomaterial allows for a controlled delivery of bioactive components within the nanocomposite, enhancing a regenerative potential thereof. Furthermore, the integration of the nanomaterials within the biomimetic nanocomposite scaffold enhances mechanical, chemical, and biological properties thereof, promoting improved cell-material interactions in the tissues.

Optionally, the nanofibers of the nanomaterial comprise same or different natural polymers. In this regard, by allowing the use of either the same or different natural polymers for the nanofibers, the technical effect is the enhancement of the biomimetic nanocomposite scaffold's versatility and adaptability. The flexibility enables tailoring the biomimetic nanocomposite scaffold's properties to specific tissue engineering requirements, allowing for a broader range of applications.

Optionally, a shape of the nanofibers is selected from at least one of: spherical, cylindrical, rod-shaped. In this regard, the method provides flexibility in choosing the shape of the nanofibers within the nanomaterial.

The technical effect is that it allows for the customization of the bio-mimetic nanocomposite scaffold's architecture and properties. Optionally, different shapes may influence factors such as surface area, mechanical strength, and interaction with cells or tissues. Optionally, by offering options like the spherical, the cylindrical, or the rod-shaped nanofibers, the method enables the optimization of the bio-mimetic nanocomposite scaffold's design.

Optionally, the nanofibers of the nanomaterial align in at least one of a radial orientation, a longitudinal orientation, a random orientation, or a combination thereof. In this regard, the radial orientation refers to an orientation of nanofibers that are aligned outward from a central point, resembling the spokes of a wheel. Optionally, the radial orientation is suitable for the given connective tissues such as fibrocartilage. Moreover, the longitudinal orientation refers to an orientation of the nanofibers aligned along a specific direction, similar to parallel lines. Optionally, the longitudinal orientation is preferable for hyaline or articular tissues. Furthermore, the random orientation refers to an orientation of the nanofibers that have no specific alignment pattern, providing versatility. Optionally, the random orientation could serve tissues with a distinct chondral arrangement. Optionally, the nanofibers may be oriented in a combination of two or more of the aforementioned orientations. For example, alternating longitudinal and radial fibers could be used for fibrocartilage. Optionally, the structure of the nanomaterial is implemented as a nanodot, a nondisk, or a nanofibre.

Optionally, the diameter of the fibers is in a range of 1-5000 nanometers and length of 10-100 micrometers. Optionally, the diameter of the fibers is in a range from 1, 500, 1000, 1500, 2000, 2500, 3000, 3500, 4000 or 4500 nanometers up to 1000, 1500, 2000, 2500, 3000, 3500, 4000, 4500 or 5000 nanometers. Optionally, the length of the fibers is in a range from 10, 20, 30, 40, 50, 60, 70, 80 or 90 nanometers up to 20, 30, 40, 50, 60, 70, 80, 90 or 100 micrometers. The technical effect of said range of the diameters and the lengths is that it offers customization and versatility in tailoring the bio-mimetic nanocomposite scaffold's nanofibers to specific tissue engineering applications.

Optionally, receiving the bio-mimetic nanocomposite printable material comprises a mixture of the bulk polymer crosslinked matrix with the nanomaterial. Optionally, the mixture is obtained by mixing or dispersing the nanomaterial throughout the bulk polymer crosslinked matrix in a homogenous manner in order to obtain a uniform bio-mimetic nanocomposite printable material. Optionally, the mixing is an active mixing, a passive mixing, or a combination of both. Optionally, the active mixing includes screw-driven mixing where energy is added through stirring, vortexing, and so forth, leading to turbulent flow patterns. Optionally, the passive mixing includes diffusion through multi-fluidic chips.

Optionally, the bio-mimetic nanocomposite printable material comprises mammalian cells selected from: fibroblasts, chondrocytes, fibrochondrocytes, primary human meniscus-derived chondrocytes, stem cells, bone marrow cells, embryonic stem cells, mesenchymal stem cells, bone marrow-derived mesenchymal stem cells, induced pluripotent stem cells, differentiated stem cells, tissue-derived cells, microvascular endothelial cells, and combinations thereof. In this regard, the method provides the technical advantage of versatility and specificity in choosing the mammalian cells to be incorporated into the bio-mimetic nanocomposite printable material. Optionally, by including various cell types such as fibroblasts, chondrocytes, and stem cells, the method enables the customization of the scaffold's composition and gradient structures to match the intended tissue regeneration application. Optionally, the bio-mimetic nanocomposite printable material further comprises active agents selected from: vascular endothelial growth factors (VEGF), fibroblast growth factors (FGF), Transforming growth factor beta factors (TGF-β), insulin-like growth factors (IGF), or within each of these growth factor families, or a combination thereof. The term “active agents” as used herein refers to substances or compounds that have a specific biological or therapeutic effect. Optionally, the active agents can include a wide range of substances, such as drugs, growth factors, hormones, enzymes, antibodies, and other bioactive molecules, that interact with biological systems to produce a therapeutic or physiological response. In this regard, the active agents are incorporated into the bio-mimetic nanocomposite printable material during its formulation, typically through controlled mixing and dispersion methods. The technical effect of incorporating the active agents is to enable the controlled integration of growth factors within the bio-mimetic nanocomposite printable material. It will be appreciated that by selecting specific growth factors such as VEGF, FGF, TGF-β, IGF, or combinations thereof, it becomes possible to fine-tune the bio-mimetic nanocomposite scaffold's regenerative properties. For example, the VEGF can stimulate blood vessel formation, while the TGF-β can promote tissue remodelling.

The term “printing surface” as used herein refers to a base or a three-dimensional (3D) substrate on which the bio-mimetic nanocomposite printable material is deposited to create the bio-mimetic nanocomposite scaffold. Optionally, the printing surface may be of various shapes and sizes. In this regard, the printing surface may have a specific shape, a cross-section, a degree of flatness and/or an angle with respect to a plane. Optionally, the printing surface is a planar surface or a non-planar surface. Optionally the printing surface can move along three axes, i.e. an x-axis (sideways (i.e. left and right direction) relative to the one or more printing heads), a y-axis (back and forth direction relative to the one or more printing heads), and a z-axis (up and down direction relative to the one or more printing heads). Optionally, the printing surface is coupled to a movement mechanism that enables the movement of the printing surface with respect to the movement of the one or more printing heads. Optionally, the printing surface may rotate with respect to the movement of the one or more printing heads. Optionally, the movement mechanism may be arranged (namely, accommodated) below the printing surface. Optionally, the movement mechanism enables a horizontal movement (along x-axis as well as y-axis) of the printing surface and a vertical movement (along z-axis) of the printing surface. Optionally, the horizontal movement of the printing surface may be achieved by the conveyor unit type implementation of the printing surface.

The method comprises dispensing the bio-mimetic nanocomposite printable material in one or more layers on the printing surface, based on the generated bio-mimetic tool path, for producing the bio-mimetic nanocomposite scaffold. Herein, the dispensing refers to the controlled release of the bio-mimetic nanocomposite printable material onto the printing surface. The one or more layers are dispensed in the single or stacked layer deposition manner at the printing surface, based the generated bio-mimetic tool path, for fabricating the bio-mimetic nanocomposite scaffold. In this regard, the method employs the one or more printing heads that operate based on the generated bio-mimetic tool path, which guides the movement and deposition of the bio-mimetic nanocomposite printable material.

In an example, for the single layer deposition, the one or more printing heads dispense a uniform layer of the material onto the printing surface, following the generated tool path. The single layer deposition is repeated for each layer, resulting in a thin and precise bio-mimetic nanocomposite scaffold. In another example, for the stacked layer deposition, the one or more printing heads release multiple layers of the bio-mimetic nanocomposite material on top of each other, building a three-dimensional scaffold. It will be appreciated that the one or more printing heads coordinates with the generated bio-mimetic tool path to ensure the layers are stacked with precision, leading to the creation of a complex and voluminous scaffold.

Optionally, the bio-mimetic nanocomposite scaffold is implemented as fibrocartilage, elastic cartilage, or hyaline cartilage. The term “fibrocartilage” as used herein refers to a type of cartilage found in the human body that has a dense network of the collagen fibers. Typically, the fibrocartilage is known for its toughness and ability to withstand compression and tension. Optionally, the fibrocartilage is typically found in structures like intervertebral discs, menisci in the knee, and some tendons.

The term “elastic cartilage” as used herein refers to a type of cartilage that contains abundant elastic fibers in addition to collagen. Optionally, the elastic cartilage is highly flexible and can return to its original shape after deformation. Optionally, the elastic cartilage is primarily found in the external ear, the epiglottis, and the larynx. The term “hyaline cartilage” as used herein refers to a cartilage having a glassy or translucent appearance and contains collagen fibers. Optionally, the hyaline cartilage is found in the nose, trachea, bronchi, and at the ends of long bones where it forms the articular cartilage that cushions joint surfaces. Optionally, when regenerating intervertebral discs or knee menisci, which experience compressive forces, the bio-mimetic scaffold mimicking the fibrocartilage properties can provide the necessary support and durability. Optionally, the compressive forces are largely mitigated by the high water content and elasticity within the native tissue, while the tensile forces are mitigated by the alignment of strong collagen fibers. Optionally, implementing the scaffold as the elastic cartilage can help recreate the natural flexibility and shape-maintaining properties of these tissues. Optionally, when regenerating joint cartilage, such as in the knee or nose, mimicking the hyaline cartilage properties is crucial for maintaining joint function and reducing friction and mitigating the mechanical forces exerted on the tissue. The bio-mimetic scaffold can be implemented as hyaline cartilage to achieve these characteristics.

Optionally, the method further comprises aligning, while dispensing the one or more layers, nanofibers of the nanomaterial of the bio-mimetic nanocomposite printable material in a direction of the bio-mimetic tool path. In this regard, during the dispensing of the one or more layers, the method enables the alignment of the nanofibers of the nanomaterial. Optionally, the aligning is facilitated using shear forces of the one or more printing heads. Optionally, the nanofibers such as stimuli-responsive materials are aligned through external factors, including but not limited to temperature, pH, ultrasound, magnetic and electrical fields. In such a case, when the external magnetic or electrical fields are used for aligning the nanofibers, then the nanofibers are positioned in a perpendicular direction to the bio-mimetic tool path. Optionally, the method further comprises aligning the nanofibers based on a design of the bio-mimetic nanocomposite scaffold. The alignment is performed in a specific direction corresponding to the bio-mimetic tool path. By aligning the nanofibers in the direction of the bio-mimetic tool path, the resulting bio-mimetic nanocomposite material exhibits enhanced structural properties. The aligned nanofibers create a hierarchical structure that closely resembles the natural arrangement of fibers found in biological tissues, such as muscles or tendons of the subject. For example, while it is sometimes hard to align the fibers during the printing process, the chemical structure of the fibers could be modified in that they become stimuli-responsive through the external factors. When printing the hyaline or the articular cartilage, the collagen fibers are oriented either perpendicular or parallel. In such a case, the collagen fibers are oriented by printing and then taking a pause in the printing and applying the magnetic field in the direction of alignment to stimulate the iron-functionalized collagen.

Optionally, the method further comprises controlling a first set of parameters associated with the multi-dimensional printer, wherein the first set of parameters are selected from: temperature of one or more printing heads; temperature of the printing surface; pressure on the one or more printing heads; and light intensity and duration, temperature and humidity, particles, VOC, gas, weight, monitoring in a printing chamber. In this regard, the method comprises controlling the aforementioned first set of parameters for ensuring the precision, quality, and effectiveness of the bio-mimetic nanocomposite scaffold. Optionally, the method comprises controlling the temperature of the one or more printing heads and the printing surface in order to optimize the bio-mimetic nanocomposite printable material deposition and the layer adhesion. It will be appreciated that the temperature regulation prevents the bio-mimetic nanocomposite printable material from clogging or warping, ensuring a consistent and uniform structure of the bio-mimetic nanocomposite scaffold. Optionally, the method comprises controlling the pressure on the one or more printing heads to determine the flow rate of the bio-mimetic nanocomposite printable material, affecting the accuracy of deposition. Optionally, the controlled pressure ensures the bio-mimetic nanocomposite printable material is dispensed at the desired rate, contributing to the scaffold's dimensional accuracy. Optionally, the temperature is in a range of 20 to 65° C. (consistent with the biopolymers). The temperature may thus be from 20, 25, 30, 35, 40, 45, 50, 55 or 60° C. up to 25, 30, 35, 40, 45, 50, 55, 60 or 65° C. Optionally, the pressure is in a range of 0 to 200 bar. The pressure may thus be from 0, 10, 20, 30, 40, 50, 60, 70, 80, 90, 100, 110, 120, 130, 140, 150, 160, 170, 180 or 190 bar up to 20, 30, 40, 50, 60, 70, 80, 90, 100, 110, 120, 130, 140, 150, 160, 170, 180, 190 or 200 bar. Optionally, the UV wavelength, intensity and duration depend on (meth) acrylation of the polymer such as in a range of 200-400 nm. The UV wavelength may thus be from 200, 210, 220, 230, 240, 250, 260, 270, 280, 290, 300, 310, 320, 330, 340, 350, 360, 370, 380 or 390 nm up to 210, 220, 230, 240, 250, 260, 270, 280, 290, 300, 310, 320, 330, 340, 350, 360, 370, 380, 390 or 400 nm.

Optionally, in processes involving light-curable materials, such as some bio-mimetic nanocomposite formulations, controlling the light intensity and duration is critical for proper curing or crosslinking. Optionally, precise control ensures that the bio-mimetic nanocomposite printable material solidifies correctly, leading to the desired mechanical properties. Optionally, monitoring and controlling factors such as the temperature, the humidity, the particles, the volatile organic compounds (VOCs), and the gas levels in the printing chamber create a stable and contamination-free environment. For example, the gas is nitrogen gas, oxygen gas, carbon dioxide gas, argon gas or ethylene oxide gas. This prevents contamination of the bio-mimetic nanocomposite printable material and maintains a suitable atmosphere for the printing process. Optionally, the printing chamber environment parameters is selected before printing. Optionally, said selection is based on the encapsulated cells to ensure optimal cytocentric conditions. Optionally, said selection is based on the crosslinking requirements of the chosen bulk polymer crosslinked matrix, including ionic, photo and chemical crosslinking, or combination thereof.

Optionally, the method further comprises controlling a second set of parameters associated with the bio-mimetic nanocomposite printable material, wherein the second set of parameters are selected from: mass, weight, volume, density, and flow speed of the bio-mimetic nanocomposite printable material, and wherein the second set of parameters is configured for providing quality control during the printing process. In this regard, the second set of parameters is essential for maintaining the consistency, quality, and performance of the bio-mimetic nanocomposite printable material throughout the printing process. Optionally, ensuring that the bio-mimetic nanocomposite printable material is dispensed in the correct quantities guarantees that each layer of the bio-mimetic nanocomposite scaffold has the desired composition.

Optionally, monitoring and controlling the density of the bio-mimetic nanocomposite printable material influences its structural integrity and mechanical properties. Optionally, maintaining consistent density is crucial for achieving the desired strength and functionality of the printed scaffold. Optionally, managing the flow speed of the bio-mimetic nanocomposite printable material through the one or more printing heads is vital for controlling the deposition rate and uniformity. Optionally, the flow speed impacts the accuracy of material placement and the overall quality of the bio-mimetic nanocomposite scaffold. Optionally, controlling the second set of parameters ensures reproducibility and decreased batch-to-batch variability.

Optionally, the method further comprises controlling one or more environmental conditions after producing the bio-mimetic nanocomposite scaffold by performing at least one of: incubating the bio-mimetic nanocomposite scaffold; controlling of temperature; controlling of carbon dioxide; controlling of oxygen, and controlling of humidity in the printing chamber. In this regard, the incubation provides a controlled environment that promotes cell attachment, proliferation, and tissue formation on the bio-mimetic nanocomposite scaffold. Optionally, the incubation aids in enhancing cell viability and functionality. Optionally, the incubation conditions can provide a platform for studying the effects of the 3D bioprinted constructs in vitro. Additionally, controlling temperature and carbon dioxide levels in the printing chamber ensures that the bio-mimetic nanocomposite scaffold and its embedded cells remain in an environment conducive to their growth and development. Optionally, optimal temperature regulation supports biochemical reactions necessary for tissue formation, while managing the carbon dioxide levels helps maintain the PH balance for cell growth. The aforementioned environmental controls contribute to the overall success and effectiveness of the bio-mimetic nanocomposite scaffold in its intended biological applications, ensuring that it fosters tissue regeneration and repair efficiently. Optionally, by maintaining specific oxygen concentrations, it's possible to create an environment that promotes or inhibits cellular growth and tissue development, crucial for mimicking natural biological systems. Moreover, controlling the humidity is essential for preserving the structural integrity of the biomimetic scaffold and ensuring proper cell attachment and proliferation. Furthermore, appropriate humidity levels prevent dehydration of the biomimetic scaffold, which is essential for the successful growth of cells and tissues within the biomimetic structure.

Optionally, the method further comprises sterilizing the bio-mimetic nanocomposite scaffold. The term “sterilizing” as used herein refers to a process of eliminating or reducing all viable microorganisms, including bacteria, viruses, and fungi, as well as their spores, from the scaffold's surface and interior. Optionally, the sterilizing procedure aims to render the scaffold completely free from any potential sources of contamination, making it suitable for safe use in medical applications such as tissue regeneration, transplantation, or implantation. Optionally, the sterilizing is performed using aseptic techniques during the additive manufacturing. Optionally, the aseptic techniques involve maintaining a sterile environment within the printing chamber and ensuring that all components of the multidimensional printer, including the one or more printing heads and the bio-mimetic nanocomposite printable materials, are kept free from contamination. Optionally, a final sterilization step may be employed after the bio-mimetic nanocomposite scaffold is produced. The final sterilization ensures that the bio-mimetic nanocomposite scaffold is sterile and ready for use in medical applications. Optionally, various sterilization methods can be utilized, including heat/steam sterilization, irradiation sterilization, chemical sterilization, or a combination thereof.

### DETAILED DESCRIPTION OF THE DRAWINGS

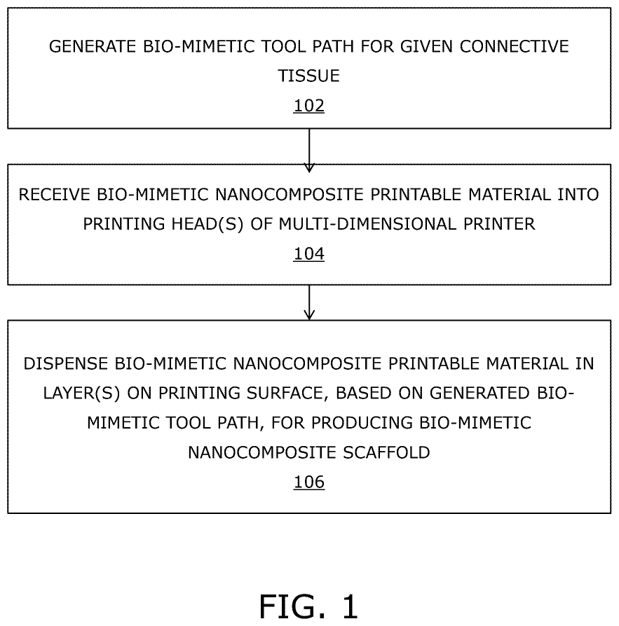

Referring to FIG. 1, illustrated is a flowchart depicting steps of an additive manufacturing method for producing a bio-mimetic nanocomposite scaffold using a multi-dimensional printer, in accordance with an embodiment of the present disclosure. At step 102, a bio-mimetic tool path is generated for a given connective tissue. At step 104, a bio-mimetic nanocomposite printable material is received into one or more printing heads of the multi-dimensional printer. At step 106, the bio-mimetic nanocomposite printable material is dispensed in one or more layers on a printing surface, based on the generated bio-mimetic tool path, for producing the bio-mimetic nanocomposite scaffold.

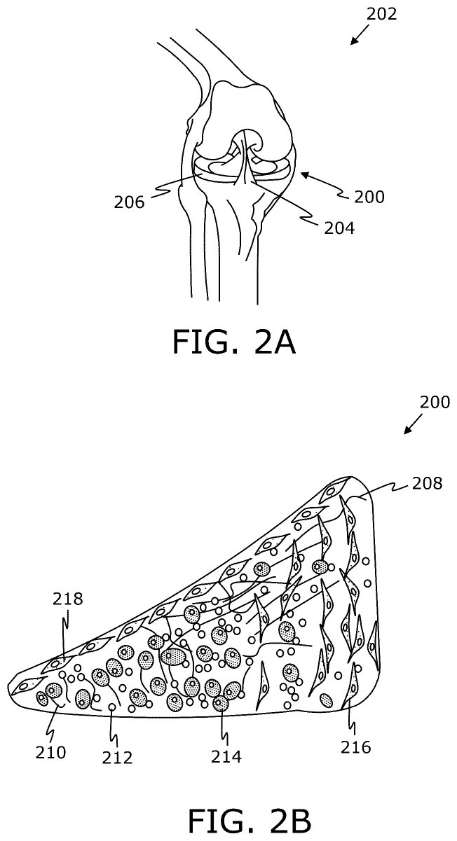

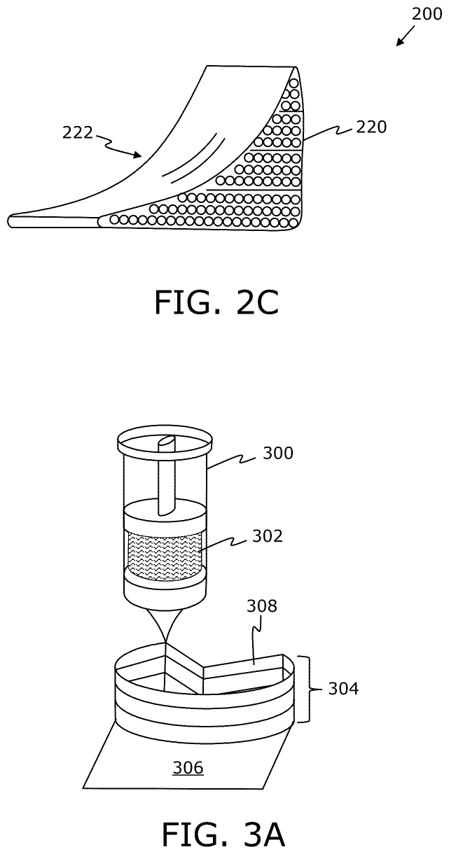

Referring to FIG. 2A, illustrated is a scan of a given connective tissue 200 of a knee 202 of a subject, in accordance with an embodiment of the present disclosure. As shown, an anatomy of a meniscus such as a medial meniscus 204 and a lateral meniscus 206 of the knee 202. Moreover, the anatomy of the meniscus is used for generating a bio-mimetic tool path by identifying a macroscopic architecture of the knee 202 or regions of interest on the knee 202. Herein, the macroscopic architecture depicts that a bio-mimetic nanocomposite scaffold for the given connective tissue 200 is implemented as a fibrocartilage.

Referring to FIG. 2B, illustrated is a scan of a microscopic architecture of the given connective tissue 200 of the subject, in accordance with an embodiment of the present disclosure. As shown, the given connective tissue 200 (such as the meniscus) comprises collagen I 208, collagen II 210, proteoglycans 212, fibrochondrocytes 214, fibroblasts 216, and fusiform cells 218.

Referring to FIG. 2C, illustrated is an orientation of nanofibers 220 in the given connective tissue 200 of the subject, in accordance with an embodiment of the present disclosure. Herein, the nanofibers 220 are collagen fibers. As shown, the nanofibers 220 are aligned in a radial orientation 222 around the circumference of the given connective tissue 200.

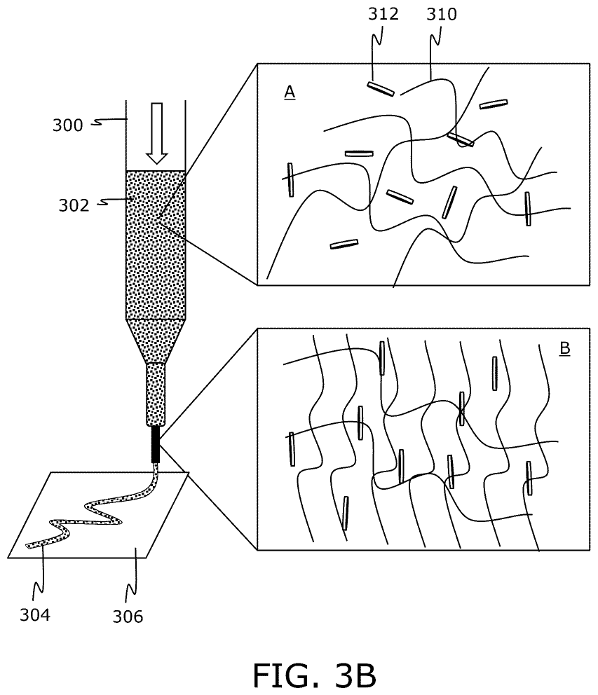

Referring to FIGS. 3A and 3B, illustrated is a printing head 300 of a multi-dimensional printer performing an additive manufacturing, in accordance with an embodiment of the present disclosure. As shown in FIG. 3A, the printing head 300 is dispensing a bio-mimetic nanocomposite printable material 302 in one or more layers 304 on a printing surface 306, based on a generated bio-mimetic tool path, for producing the bio-mimetic nanocomposite scaffold 308.

As shown in FIG. 3B, the bio-mimetic nanocomposite printable material 302 comprises a mixture of a bulk polymer crosslinked matrix 310 with a nanomaterial 312 to form the bio-mimetic nanocomposite printable material 302. Moreover, as shown, while dispensing the one or more layers 304 the nanofibers of the nanomaterial 312 of the bio-mimetic nanocomposite printable material 302 are aligned in a direction of the bio-mimetic tool path. At point A, there is shown that the nanofibers of the nanomaterial 312 are aligned in a random orientation. At point B, there is shown that the nanofibers of the nanomaterial 312 are aligned in a longitudinal orientation and a parallel orientation.

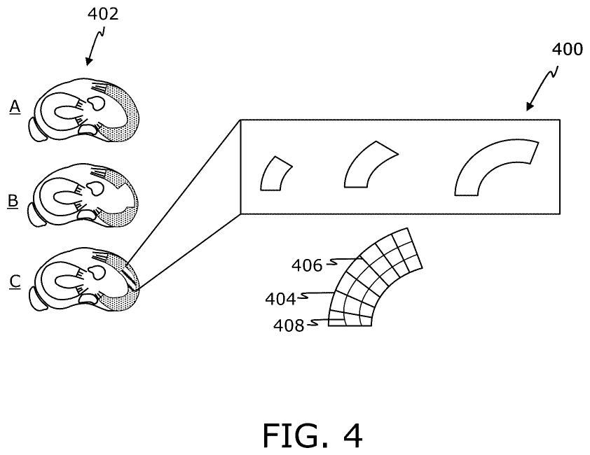

Referring to FIG. 4, illustrated is a process of generating a bio-mimetic tool path 400 for a given connective tissue 402, in accordance with an embodiment of the present disclosure. At point A, the given connective tissue 402 such as a meniscus of a knee of a subject is shown. At point B, the given connective tissue 402 is shown in an injured condition. At point C, the bio-mimetic tool path 400 is generated for producing a bio-mimetic nanocomposite scaffold in order to repair the given connective tissue 402. There is shown, the bio-mimetic tool path 400 considering a microscopic architecture 404 of the given connective tissue 402. Moreover, the microscopic architecture 404 includes alternating infill patterns of a longitudinal orientation 406 and a radial orientation 408 of nanofibers of a bio-mimetic nanocomposite printable material.

FIGS. 2A, 2B, 2C, 3A, 3B and 4 are merely examples, which should not unduly limit the scope of the claims herein. A person skilled in the art will recognize many variations, alternatives, and modifications of embodiments of the present disclosure.

1. An additive manufacturing method for producing a bio-mimetic nanocomposite scaffold using a multi-dimensional printer, the method comprising:
generating a bio-mimetic tool path for a given connective tissue;
receiving a bio-mimetic nanocomposite printable material into one or more printing heads of the multi-dimensional printer; and
dispensing the bio-mimetic nanocomposite printable material in one or more layers on a printing surface, based on the generated bio-mimetic tool path, for producing the bio-mimetic nanocomposite scaffold.

2. A method according to claim 1, wherein generating the bio-mimetic tool path comprises performing at least one of: an anatomical sectioning of given connective tissue using a biopsy, a real-time imaging using collagen auto-fluorescence.

3. A method according to claim 1, wherein receiving the bio-mimetic nanocomposite printable material comprises a mixture of a bulk polymer crosslinked matrix with a nanomaterial to form the bio-mimetic nanocomposite printable material.

4. A method according to claim 1, further comprising aligning, while dispensing the one or more layers, nanofibers of the nanomaterial of the bio-mimetic nanocomposite printable material in a direction of the bio-mimetic tool path.

5. A method according to claim 1, further comprising controlling a first set of parameters associated with the multi-dimensional printer, wherein the first set of parameters are selected from: temperature of one or more printing heads; temperature of the printing surface; pressure on the one or more printing heads; and light intensity and duration, temperature and humidity, particles, VOC, gas, weight, monitoring in a printing chamber.

6. A method according to claim 1, further comprising controlling a second set of parameters associated with the bio-mimetic nanocomposite printable material, wherein the second set of parameters are selected from: mass, weight, volume, density, and flow speed of the bio-mimetic nanocomposite printable material, and wherein the second set of parameters is configured for providing quality control during the printing process.

7. A method according to claim 1, further comprising controlling one or more environmental conditions after producing the bio-mimetic nanocomposite scaffold.

8. A method according to claim 1, further comprising sterilizing the bio-mimetic nanocomposite scaffold.

9. A method according to claim 1, wherein the bulk polymer crosslinked matrix comprises: collagen, gelatin, laminins, chitosan, agarose, alginate, fibronectin, cellulose, glycosaminoglycans (GAG), deoxyribonucleic acid (DNA), adhesion glycoproteins, elastin, or a combination thereof.

10. A method according to claim 9, wherein the collagen is selected from: collagen 0, collagen I, collagen II, collagen III, collagen IV, collagen V, collagen VI, or collagen XVIII.

11. A method according to claim 9, wherein the glycosaminoglycans are linear polysaccharides consisting of repeating disaccharide units, including but not limited to hyaluronic acid, chondroitin-6-sulfate, chondroitin-4-sulfate, or keratin-sulphate.

12. A method according to claim 1, wherein the nanofibers of the nanomaterial comprise same or different natural polymers.

13. A method according to claim 1, wherein a shape of the nanofibers is selected from at least one of: spherical, cylindrical, rod-shaped.

14. A method according to claim 1, wherein the nanofibers of the nanomaterial align in at least one of a radial orientation, a longitudinal orientation, a random orientation, a parallel orientation, a perpendicular orientation, an oblique orientation, a wound orientation or a combination thereof.

15. A method according to claim 1, wherein the diameter of the fibers is in a range of 1-5000 nanometers and length of 10-100 micrometers.

16. A method according to claim 1, wherein the bio-mimetic nanocomposite printable material comprises mammalian cells selected from: fibroblasts, chondrocytes, fibrochondrocytes, primary human meniscus-derived chondrocytes, stem cells, bone marrow cells, embryonic stem cells, mesenchymal stem cells, bone marrow-derived mesenchymal stem cells, induced pluripotent stem cells, differentiated stem cells, tissue-derived cells, microvascular endothelial cells, and combinations thereof.

17. A method according to claim 1, wherein the bio-mimetic nanocomposite printable material further comprises active agents selected from: vascular endothelial growth factors (VEGF), fibroblast growth factors (FGF), Transforming growth factor beta factors (TGFB), insulin-like growth factors (IGF), platelet-derived growth factors (PDGF) or within each of these growth factor families and super-families, or a combination thereof.

18. A method according to claim 1, wherein the bio-mimetic nanocomposite scaffold is implemented as fibrocartilage, elastic cartilage, or hyaline/articular cartilage.
